(19) United States
(12) Patent Application Publication
(10) Pub. No.: US 2009/0132047 A1
(43) Pub. Date: May 21, 2009

(54) ANCHORING SYSTEMS AND INTERFACES FOR FLEXIBLE SURGICAL IMPLANTS FOR REPLACING CARTILAGE

(76) Inventors: Kevin A. Mansmann, Paoli, PA (US); Alvin A. Potter, Glen Mills, PA (US)

Correspondence Address: PATRICK D. KELLY, 11939 MANCHESTER #403, ST. LOUIS, MO, 63131, US

(21) Appl. No.: 12/067,654

(22) PCT Filed: Nov. 30, 2005
(86) PCT No.: PCT/US05/43444

§ 371 Date: Oct. 27, 2008

Related U.S. Application Data
(60) Provisional application No. 60/656,606, filed on Feb. 26, 2005.
(60) Provisional application No. 60/631,652, filed on Nov. 30, 2004.

(51) Int. Cl. A61F 2/08; A61F 2/02; A61B 17/58

(52) U.S. Cl. 623/14.12; 623/11.11; 606/300

(57) ABSTRACT

Surgical implants for replacing cartilage are provided with hydrogel polymers affixed to anchors made of “shape-memory” materials, such as nitinol alloys. These implants can be flexed, allowing them to be inserted into joints arthroscopically. After insertion, an implant will return to its manufactured size and shape, and can be anchored to bone or other tissue. The anchoring components can grip and hold hydrogels or other soft polymers by means of an interface of porous fabric. The fabric can support a reinforcing mesh embedded within the soft polymer, and its bottom surface can promote tissue ingrowth, leading to stronger anchoring. Two or more porous layers can enclose a soft polymer, for purposes such as sustained drug release or holding transplanted cells.

### RELATED APPLICATION

This application is a national counterpart of Patent Cooperation Treaty application PCT/US2005/043444, filed Nov. 30, 2005, which claimed priority based on U.S. provisional applications 60/631,652 (filed Nov. 30, 2004) and 60/656,606 (filed Mar. 25, 2005).

### FIELD OF THE INVENTION

This invention is in the field of medicine and surgery, and relates to surgical implants that require anchoring systems, such as hydrogel implants for repairing or replacing cartilage in a knee, shoulder, or other joint.

### BACKGROUND OF THE INVENTION

Background information on surgical implants that can be used to replace damaged cartilage, in mammalian joints such as knees or hips, is available in various books, patents, and articles that are cited and discussed in several prior patent applications by the first-named inventor herein, Kevin Mansmann, an orthopedic surgeon. Those applications include several applications published under the Patent Cooperation Treaty (PCT) system, and several other applications published on the US Patent and Trademark Office website.

In particular, PCT publication WO 03/103543 (arising from PCT/US02/09486) describes flexible implants having at least one articulating surface made of a smooth and wettable (“lubricious”) polymer, and at least one “anchoring” surface. The “articulating” surface will press, rub, and slide against another cartilage surface in a joint such as a knee, hip, etc., while the anchoring surface enables the implant to be securely and permanently affixed to hard bone or other tissue.

These types of implants can be divided into two categories, which are: (1) meniscal-type implants, having a wedge-shaped cross-section, designed to be anchored to soft tissue such as the tendons and ligaments that surround and enclose the synovial fluid in a knee joint; and, (2) condylar-type implants, designed to be anchored directly to a hard bone surface, such as a femoral runner, tibial plateau, or patella, in a knee joint, or to the surfaces of a ball-and-socket joint in a hip or shoulder.

It should be noted that the terms condyle and condylar are not always used consistently. Some people limit those terms to the rounded ends of elongated bones, while others use them to refer to any bone surface covered with hyaline cartilage, which is affixed to bone via a transitional (subchondral) layer that is interlineated with collagen fibers that penetrate into both bone and cartilage, providing a three-dimensional anchoring gradient, rather than a simple planar interface. As used herein, condyle or condylar refer to any bone surface covered by cartilage, and condylar implants include any implants designed to be affixed directly to a surface on a bone.

Currently, most devices used as knee, hip, or shoulder implants have one piece with a hard and impermeable plastic surface (usually made from a polymer such as ultra-high-molecular-weight polyethylene, abbreviated as UHMWPE), which presses and slides against a second piece that has a hard metal surface (usually made from a titanium steel alloy). These implants are large and inflexible, and they can be inserted into a joint only after relatively large segments of bone have been removed, in operations that are referred to as “open joint” operations, to distinguish them from “arthroscopic” or “minimally invasive” surgery. In any “open joint” operation, extensive damage must be inflicted on the surrounding tissues (including muscles, tendons, and ligaments) and blood vessels (also called the vasculature).

Arthroscopic operations (which are a subset of surgical operations) inflict much less damage, and are preferred whenever possible. However, under the prior art, arthroscopic replacement of large cartilage segments in joints such as knees, hips, or shoulders has not been able to create repairs that can last reliably for years or even decades; therefore, such operations are not being done.

The invention disclosed herein is one in a series of steps that will render complete arthroscopic repair of even large joints (such as knees, hips, and shoulders) feasible and practical. Rather than using hard impermeable plastic pieces that will rub against steel alloys, this line of research focuses on the use of relatively thin and flexible segments of specialized polymers called hydrogels. Hydrogels allow water molecules to travel and permeate through a three-dimensional lattice of crosslinked polymeric chains.

Hydrogels are of interest, partly because they're flexible (which makes them well-suited for arthroscopic insertion, when rolled up into a cylindrical configuration that can slide through a minimally-invasive incision, such as by using an insertion tube), and partly because most types of soft tissue (including cartilage) are hydrogels. The body is an adaptive system, and if a broken part can be replaced by an artificial part having a similar structure, the remainder of the body can adapt to the replacement part more easily and readily than it can adapt to a completely different type of substitute. As an analogy, even though wheels can be extraordinarily useful, no one who loses a leg ever has it replaced by a prosthetic leg with a wheel at the bottom. Instead, prosthetic legs are designed to behave and perform in ways that generally emulate the normal structure, behavior, and performance of a natural leg.

By the same token, if a synthetic implant is designed to replace damaged cartilage (references to “damaged” cartilage are used broadly herein, and include damage due to any causative or aggravating factor, such as trauma or injury, a disease or disorder such as chondromalacia or arthritis, gradual wear over the course of a lifetime, lack of proper nutrition, etc.), the implant can be made in the same size and shape as a layer of cartilage, and if it can perform as a hydrogel that will simply replace the hydrogel of native cartilage with as little disruption as possible, lower levels of stress and damage will be inflicted on the surrounding tissues, compared to cutting open a joint, sawing out segments of bone several inches long, and replacing the bone with large pieces made of steel and hard plastic.

However, despite those factors, hydrogels have not been used to replace hyaline cartilage in load-bearing joints, because they are not as strong and durable as other known types of dense and impermeable plastic, such as UHMWPE. Hydrogels must contain large numbers of open spaces and tunnels in their molecular structures, to allow water molecules to permeate through the polymeric matrix in a relatively free and rapid manner. Since water takes up a substantial part of the volume but cannot impart any strength, a hydrogel is not as strong as a plastic that is entirely filled with densely-crosslinked chains.

Therefore, to develop improved hydrogels that are strong and durable enough to replace hyaline cartilage, even in load-bearing joints such as knees, methods and materials are being developed, as described in PCT application WO 03/103543, in which three-dimensional meshes (also called matrices or similar terms) made of very strong fibers are being used to reinforce synthetic hydrogels. Such meshes must have substantial thickness (i.e., more than can be provided by a single layer of conventional material that is woven or knitted, even if relatively thick yarn is used to make the material); however, the mesh cannot be exposed on the smooth articulating surface of a hydrogel implant designed to replace hyaline cartilage, since an exposed mesh surface would cause abrasion, leading to damage.

Polymers that can be made into strong and durable hydrogels include, for example, polyacrylonitrile (PAN), and polyurethane. Either of those two classes of polymers can allow various types of optional molecular groups to be incorporated into the backbone chains, the “side groups” or “side chains” that are attached to backbone chains, and/or any crosslinking bonds or chains that are used to bond the backbone chains to each other. To form hydrophilic and flexible gel versions of either PAN (which is more widely recognized by the trademark name ORLON™) or polyurethane, “polar” groups that will attract water molecules (which also are polar) can be incorporated into the “monomer” compounds that are used to manufacture the final polymers. These molecular factors, and reagents for manufacturing such polymers, are well known to polymer chemists.

An additional PCT application, published as WO 05/032426, discloses two more advances in the development of synthetic hydrogel implants that can provide strong and durable replacements for hyaline cartilage, in load-bearing joints.

One disclosure involves using sulfur compounds or similar reagents to create a negative electrical charge on the articulating surface of a hydrogel implant. This negative charge should have a charge density comparable to the negative charge on healthy natural cartilage surfaces (the “fixed charge density” (FCD) of human cartilage ranges from about −50 to −250 millimolar (mM), depending on the age of the person, the location of the cartilage, and the status and condition of the cartilage). This negative charge helps cartilage interact with positively-charged components of synovial fluid (the fluid that keeps cartilage surfaces wet and lubricated, in a joint). Samples of synthetic PAN polymers that were surface-treated in this manner showed substantially improved performance, in wear tests using a machine called a tribometer.

The second disclosure involves an improved approach to anchoring a condylar implant to a bone surface, using arthroscopic methods. In this approach, several holes are drilled into a bone surface, and externally-threaded anchoring sleeves (which also can be called barrels, cylinders, or similar terms) are emplaced in the holes. Subsequently, a flexible implant is inserted (such as in a rolled-up form, through an arthroscopic insertion tube) into the joint that is being repaired. After the implant is inside the joint, it is unrolled and positioned, and pegs that are affixed to the bottom side (i.e., the anchoring surface) of the implant are pushed into the anchoring sleeves that were installed in the bone. This approach keeps the implant out of the joint and out of the way, until after a surgeon has prepared and drilled the bone surface, and has secured the anchoring sleeves in their proper locations.

After the filing of PCT application WO 05/032426 but before its publication, it was realized by the inventors herein that a certain class of metal alloys, usually referred to as “nitinol” or “shape-memory alloys”, may allow various enhancements to be provided in the design, construction, and use of implants for replacing cartilage. Those enhancements are the subject of this invention; accordingly, background information needs to be provided on nitinol and other shape-memory alloys.

These alloys, which are exceptionally strong and much more elastic than most other metals used in surgical instruments or implants, will be preferred for surgical implants that will be subjected to relatively heavy “loading” and stresses, such as in a knee joint. However, in other types of joints (such as finger joints, for example), the stresses that will be encountered are much smaller. Accordingly, rims and anchoring devices made of flexible plastic or other materials may be entirely adequate, for at least some such uses in joints that will not be heavily loaded. Accordingly, nitinol and other shape-memory alloys are discussed and described herein as exemplary and illustrative, rather than limiting and exclusive.

In effect, these alloys can be used to create strong and durable yet highly flexible implants that can be used to solve some of the most difficult problems that confront orthopedic surgeons and their patients. After these devices have been disclosed, it will be apparent to those skilled in the art how these designs and materials can be adapted to create other devices that can address lesser challenges, in joints that do not need to withstand heavy loadings and stresses.

The earliest shape-memory alloys (first identified in the 1930's) contained mainly nickel and titanium. As a result, the term “nitinol” (pronounced NIGHT-in-all) was coined as a semi-acronym (or spliced word) that combines nickel, titanium, and “NOL” (the acronym for “Naval Ordnance Laboratories”, the U.S. federal research center where nitinol's properties were discovered). During the decades that followed, other shape-memory alloys were developed with other ingredients. Even though nickel and/or titanium may not be present in some of those formulations, “nitinol” is still widely used (and is used herein) as a common name for any “shape-memory alloy” (which also can be referred to by the acronym SMA).

Briefly, nitinol alloys can go back and forth, an unlimited number of times with no deterioration, between two different states. The transition occurs when the alloy is heated above, or cooled below, a “transition temperature”. Early forms of nitinol had a transition temperature of about 70° C., which is about halfway between body temperature and the boiling point of water. That transition temperature was too high to allow safe medical use, so researchers developed different alloys with lower transition temperatures, including (for some alloys) transition temperatures of about 30° C., which is lower than body temperature.

Researchers also were able to create new formulations that reversed the temperature-dependent behaviors of the earliest nitinol alloys. In the first nitinol alloys that were discovered, the alloys would shrink when heated to a temperature above the transition temperature. That can be very useful in many situations, since contractions can be created and controlled by passing a current through the alloy, causing the alloy to heat up. Accordingly, in fields such as robotics, nitinol wires are often called “muscle wires”, since they contract and become shorter when current is applied to them, in a manner comparable to muscles in animals.

However, later-developed alloys were discovered and created that display the opposite performance traits. These alloys will shrink and contract when chilled below the transition temperature, and they will expand when warmed up above their transition temperature.

Nitinol alloys that shrink when chilled are used in a number of types of medical and surgical devices. For example, a device such as a stent, basket, or filter can be placed at the end of a catheter, cannula, or other tubular device that will be inserted into a patient through a large artery or vein. The alloy device will be kept chilled, during insertion, by pumping cold saline solution through the tube, as it passes through the blood vessel. After the device has reached a target location, the pumping of the cold solution is turned off, and the device is allowed to warm up to body temperature, causing it to expand into its final size and shape. If it is a device such as a stent, it may be detached from the insertion device, and left in the patient's body permanently. Alternately, if it is a basket or filter-type device, it may be used to “catch” a solid mass (such as a large plaque deposit, a blood clot, etc.) that is being dislodged and removed from inside an artery, so that the mass can be removed from the patient's body when the shape-memory device is cooled again, withdrawn through the artery or vein, and removed.

As mentioned above, various nitinol-type alloys are much more flexible and elastic than stainless steel and other metals used in surgery. This allows nitinol alloys to be used in various situations where flexibility and elasticity can be useful, either with or without temperature-related manipulations. As examples, various types of needles, probes, catheters, and other devices made of nitinol-type alloys can be inserted into a blood vessel, tumor, or other tissue, while forced into a relatively linear shape inside an insertion tube. After the tip of the insertion tube has reached a target location (which can be seen on fluoroscopes or other imaging devices that provide “live” images on a monitor screen), the nitinol component inside the tube is extended, until it emerges from the tip of tube. The springy elastic device that emerges from the end of the tube can expand and/or travel into any manufactured configuration, such as into a curving needle, a stent-type basket, a blood-vessel-occluding device, or “ablation” electrodes that can emit lethal microwave radiation into cells that need to be killed, such as cancer cells, or heart cells that are causing a cardiac arrhythmia.

These types of uses, in surgery and medicine, are described in more detail in various articles, such as D. Stoeckel, “Nitinol Medical Devices and Implants”, presented at the SMST 2000 Conference, and available from websites such as www.nitinol.info/pdf_files/stoeckel_{—}1.pdf. More information on shape-memory alloys is available in other sources, including full-length books such as Otsuka and Wayman, editors, Shape Memory Materials (Cambridge Univ Press, 1999), and the website of an organization called Shape Memory and Superelastic Technologies (SMST), www.smst.org.

It should be noted that, as used herein, “shape-memory” alloys or other materials must seek to return to a certain shape (which will be determined by the manufacturing process), after any deforming stresses have been released or otherwise removed. This distinguishes “shape-memory” materials from various other types of elastomers. By way of example, a rubber band is elastic, and it will return to a certain length, after any tension that caused it to take an elongated shape has been removed. However, a typical rubber band will not attempt to return to a certain specific shape; for example, if dropped onto a flat surface, it can come to rest in a relatively straight or oval-like configuration, or it can curve in either a right or left direction, without any substantial stresses arising within the rubber that makes the rubber band. By contrast, as suggested by the name, a “shape-memory” material will have a predetermined shape that was created during a manufacturing operation (which can include any annealing, curing, treating, or other shape-imparting or shape-modifying steps), and it will seek to return to that predetermined shape. This does not imply that the device must and will always return to exactly its manufactured shape; nevertheless, it will seek to do so, and any shape alterations that may be imposed by external mechanisms or forces (such as anchoring pins, an adhesive that is used to bond the material to another surface, etc.) will create some level of internal stresses within the shape-memory material. Accordingly, proper design of a device that is made of a shape-memory material must take into account the exact final shape that the device will be forced to take, once it has been installed in a particular operating environment or other destination. Some such devices are deliberately intended to create and impose mechanical forces on other components that surround it (this is comparable to installing a spring-loaded device inside a mechanism); however, if that is not the intent of a particular type of device made of a shape-memory material, then manufacture of the device should ensure that the nonstressed manufactured shape of the device is as close as possible to the final shape that the device will take after it has been installed and anchored or otherwise affixed to its final operating environment.

This current invention extends and adapts the prior teachings summarized above, into a new and different area of surgical use. This new field of use relates to nitinol (or other shape-memory alloy) components that will grip, secure, and anchor other flexible components made of completely different types of materials (such as, for example, woven layers that will both (i) encourage bone or other tissue ingrowth, to form a stronger anchoring bond after implantation, and (ii) support a three-dimensional fibrous mesh that can reinforce a synthetic polymeric hydrogel). By creating and using these types of “composite” devices (i.e., devices made of components having different materials with different physical and performance traits), surgical implants can be created with combinations of highly advantageous properties. For example, the implants disclosed herein can use anchoring components made of shape-memory alloys (or, for some devices, analogous types of flexible plastics) to provide solid, secure and durable anchoring of an implant to hard bone or soft tissue, while other components of the implant can be made of very different materials, such as soft polymeric hydrogels.

It should be noted that, in implants that will be subjected to loadings and stresses, a relatively soft material (such as a hydrogel) should not be attached directly to a much harder material (such as an anchoring rim made of hard plastic or metal). Even if an adhesive compound can firmly attach a hydrogel to a hard surface, a simple flat or rounded interface would not last very long after implantation into a loaded and stressed joint such as a knee. The stresses that will be imposed on the device will tend to focus on the interface between the soft and hard materials, and those forces and stresses eventually will push and tear the softer material off of the harder material, using “shearing” forces. Therefore, alternate designs, such as the designs disclosed below, need to be used to create durable interfaces and attachments that can couple a soft hydrogel polymer to a hard metallic or plastic surface, in a manner that can last for years or decades, even in a heavily loaded and stressed joint.

Accordingly, one object of this invention is to disclose enhanced approaches and designs for firmly and permanently securing a cloth or other fibrous layer or membrane (or other porous and flexible interface, such as an anchoring layer) to a flexible device, such as a rim made of a shape-memory alloy that surrounds (or otherwise provides structural support for) a surgical implant.

Another object of this invention is to disclose enhanced approaches and designs for firmly and permanently securing a fiber-reinforced polymeric component (such as a hydrogel polymer) to a flexible rim component (such as a rim made of a shape-memory metal alloy), in a surgical implant that will be anchored or otherwise affixed to bone or tissue.

Another object of this invention is to disclose an improved design for a class of surgical implants that can be used to replace cartilage in articulating joints.

These and other objects of the invention will become more apparent through the following summary, drawings, and detailed description.

### SUMMARY OF THE INVENTION

Methods, devices, and materials are disclosed for surgical implants that use rims or other anchoring components made of “shape-memory” materials, such as nitinol or similar alloys, to support and anchor softer polymers. The “shape-memory” material will allow an anchoring device to be flexed and stressed, by applying mechanical or similar pressures, in ways that will allow an implant to be inserted into a joint or other body part via a minimally-invasive incision (such as by using an arthroscopic insertion tube). This will allow an implant to be inserted into the body in a compacted shape that will minimize any damage to tissues that surround the insertion pathway. After insertion, the implant will expand back into its normal and unstressed size and shape, and it can be solidly anchored to a bone or other tissue. After implantation, the softer polymer can perform a desired medical function; for example, reinforced hydrogel polymers can be used to replace damaged cartilage, in a mammalian joint.

Anchoring devices made of shape-memory materials can be designed in ways that will securely grip and hold other types of materials, such as rubbery elastomers that surround and are bonded to woven porous layers of material. The porous woven material can provide an anchoring layer for an implant; this layer will promote the ingrowth of bony, scar, or other tissue into the porous layer, leading to strong permanent anchoring of an implant. The porous woven layer also can securely support a three-dimensional mesh that can reinforce a soft polymer, such as a hydrogel that can perform replace damaged cartilage; alternately, two or more porous layers, affixed to a shape-memory anchoring device, can surround and enclose a soft material, such as a polymer that provides sustained drug release, or that protects and nurtures transplanted cells.

These types of shape-memory anchoring devices can have varying stiffness and flexibility levels, and various physical designs. For example, a device can be provided by a molded “apron” component containing multiple perforations, to encourage tissue ingrowth into the apron. The apron component can contain an embedded rim component made of a shape-memory material, to provide improved anchoring, temperature-responsive, or other performance traits.

Such implants can use components that are inserted sequentially. For example, threaded anchoring receptacles can be driven into holes that have been drilled into a hard bone surface, before a flexible implant with an anchoring rim and pegs is inserted into the joint.

### BRIEF DESCRIPTION OF THE DRAWINGS

FIG. 1 is a perspective view with a partial cutaway section, showing, in sequence from the top to the bottom: (i) a hydrogel layer with a smooth and wet articulating surface, for replacing a cartilage surface; (ii) a fibrous reinforcing mesh, embedded within the hydrogel but not exposed on the smooth surface; and (iii) a layer of porous material on the anchoring surface, to promote tissue ingrowth into the implant, to provide stronger anchoring. This figure also shows an anchoring ring that surrounds the periphery of the implant, and an anchoring peg with a sawtooth surface that will fit into an anchoring sleeve.

FIG. 2 is a perspective view, from a “bottom” viewpoint, of a cartilage-replacing implant having a shape-memory rim, two anchoring pegs (shown surrounded by externally-threaded anchoring receptacles), and a porous anchoring layer that will promote tissue ingrowth into the implant after installation.

FIG. 3 is a perspective view of the shape-memory rim and anchoring components of the implant of FIG. 1, from an upper angle.

FIG. 4 is a perspective view, from a top angle, of a rim made of a shape-memory material, showing rings for holding the anchoring pegs, holes for stitching a porous anchoring fabric to the rim, and a groove around the top surface of the rim.

FIG. 5 is a perspective view, from a bottom angle, of a cartilage-replacing implant having four anchoring pegs affixed to a shape-memory rim, and an internal anchoring peg affixed to the porous anchoring fabric that is supported by the rim.

FIG. 6 is a perspective view from an upper angle, showing the internal anchoring peg affixed to the porous anchoring fabric in a manner that creates a depression or “dimple” that will not jeopardize the hydrogel layer that will be coated into the top surface of the anchoring layer.

FIG. 7 is a perspective view of an internal anchoring peg, showing slots in the upper rim that will hold two staples that will affix the peg to a fabric layer, and showing external ridges on the barrel of the peg which will interact in a ratcheting manner with similar ridges on the internal surfaces of the anchoring receptacles, to lock the pegs into the receptacles.

FIG. 8 is a perspective view of a staple that can be used to secure an internal peg to a fabric layer.

FIG. 9 is a cross-sectional perspective cutaway view of a rim of an implant for cartilage replacement, showing a shape-memory alloy tube that partially encloses a flexible rubbery polymer insert. The polymer grips and secures a fibrous fabric and/or mesh layer that will reinforce a hydrogel polymer (not shown).

FIG. 10 is a cross-section view depicting an insertion tool that will be used to force a fabric layer (wrapped around the tip of the tool) into a slot in a rubbery polymer which is mostly enclosed within a tubular shape-memory rim of an implant. A “lock ring” (also made of a shape-memory material) can be positioned at the lower tip of the tool; the lock ring will be pushed into the enlarged vacancy at the bottom of the slot in the rubbery insert, and it will remain inside the rim after assembly is complete.

FIG. 11 is a perspective view of a shape-memory rim component, which: (i) is open around its periphery, to accommodate a rubbery polymer insert that will grip and secure a porous anchoring fabric; (ii) has a plurality of slots passing through its interior surface, to enable anchoring devices to be secured to the rim; and, (iii) has a narrowed thickness at the two ends of the extruded tubular ring, to accommodate a cylindrical collar that will secure the ends of the rim to each other.

FIG. 12 is a perspective cutaway view of an anchoring device, showing: (i) an externally-threaded anchoring sleeve, which will be emplaced in a hole that has been drilled into a bone surface, before the implant is inserted into the joint; (ii) a “split stud” anchoring peg, having a cylindrical barrel that will be pushed into the anchoring sleeve until sawtooth surfaces on the barrel and the sleeve engage and lock together; (iii) a flexible Y-shaped retainer clip that will help secure the anchoring peg to the implant rim; and, (iv) a retaining staple, which will help secure the fabric to the rubbery polymer insert held by the rim.

FIG. 13 is a perspective view with a partial cutaway of a “button” implant support, for repairing a small defect in cartilage. The support has a perforated “apron” affixed to a single anchoring peg in the center, and the rim contains an embedded ring of shape-memory material. A reinforcing mesh will be affixed to the apron, and a hydrogel material will be molded around the mesh and the apron.

FIG. 14 is a perspective cutaway view of a larger implant support, showing a set of anchoring pegs around the periphery of a “perforated apron” support. This support is shaped as an open hoop, to allow a flexible anchoring fabric and a flexible fibrous mesh to provide highly flexible reinforcement for a hydrogel layer, which will be molded onto the upper surface of the implant. Alternately, the perforated apron can span the entire area of the support, and one or more additional anchoring pegs can be provided at or near the center. The outer rim of the support encloses a ring made of a shape memory material.

### DETAILED DESCRIPTION

As summarized above, this invention discloses new designs for flexible surgical implants that can be used for minimally-invasive replacement of damaged cartilage in mammalian joints, such as knees, shoulders, etc. These implants use combinations of: (1) at least one first part made from a shape-memory material, which will be used for purposes referred to herein as “anchoring” (other terms, such as securing, affixing, stabilizing, etc., can be used if desired); and, (2) at least one second part, made from a polymer material that has a desired performance trait.

In the embodiments discussed herein, replacement of damaged cartilage is the primary purpose of the implants. Accordingly, the polymer material used in such implants preferably should comprise a synthetic hydrogel that has been given a negative electrical charge on its surface, to emulate the natural charge density of natural and healthy cartilage. However, the teachings herein can be adapted for creating polymeric implants for other uses as well (such as, for example, for protecting and nurturing transplanted cells, or for sustained release of drugs).

As mentioned above, these implants are designed with a primary goal of enabling arthroscopic replacement of cartilage in knees. Knee repairs pose major challenges (due to the stresses and loadings that are imposed on knees), and due to the relative accessibility of the cartilage in knees (as compared to hip joints), initial research will involve cartilage segments in knees. After the devices disclosed herein have been developed to a point that indicates successful use in knees, those skilled in the art will then begin focusing on other joints, including hips, shoulders, fingers, wrists, ankles, etc., and the teachings herein can be adapted for use in such joints.

Similarly, the embodiments discussed herein relate to “condylar” implants, which include cartilage-replacing implants that will be secured directly to hard bone surfaces. However, the teachings herein also can be adapted for use in meniscal or labral implants, which in most cases will not require anchoring to a hard bone surface.

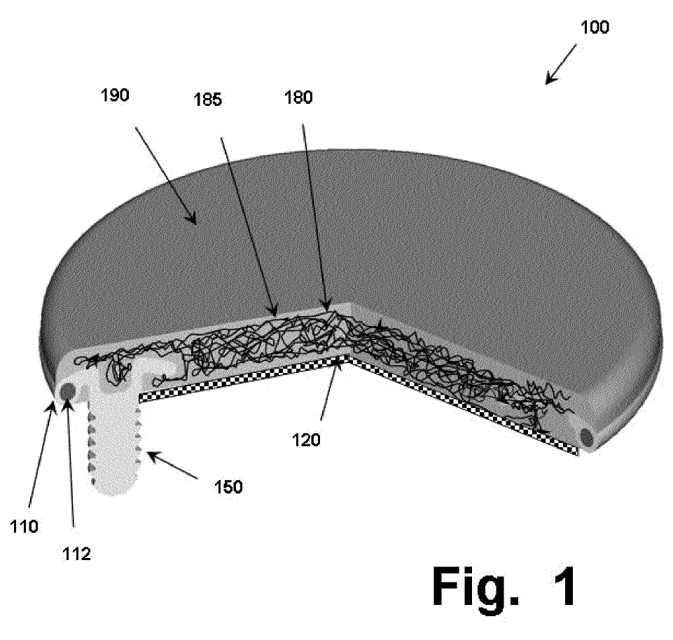

Accordingly, FIG. 1 provides a perspective view (with a partial cutaway section) of a surgical implant 100, designed for replacing hyaline cartilage in a joint such as a knee. For convenience, the smooth and wettable surface 190 is sometimes referred to herein as the “top” or “upper” surface; more accurately, it is an “articulating” surface, which will press, rub, and slide against another smooth and wettable surface of another cartilage segment (or, in most cases, another cartilage-replacing implant, since damage to a segment of cartilage in a joint inevitably leads to loss of smoothness, leading to abrasion of the surface of the other cartilage segment that rubs against the initially-damaged surface). The opposite side of the implant, shown as the lower or bottom surface in FIG. 1, is also referred to as the anchoring surface.

In the embodiment shown in FIG. 1, implant 100 is surrounded, around its entire periphery, by a molded polymeric rim 110, which encloses a reinforcing ring 111 that is made of nitinol or a similar shape-memory metal alloy. The polymer used to make rim 110 can have its own elastomeric and shape-memory traits, if desired, which will act in combination with the shape-memory behavior of metal ring 111, to achieve the desired effects and results.

The purpose of providing an anchoring component made of one or more shape-memory materials can be understood, by recognizing the series of steps (and shapes) that the anchoring component will pass through, as it is manufactured, and then surgically used. Initially, it is manufactured in a “nondeformed” and non-stressed shape and size, which will be established by the manufacturing process. The “nondeformed” shape and size refers to the shape and size that an anchoring device will take and assume, when it is not being externally stressed or deformed (for example, if the implant is allowed to simply rest on top of a flat surface, it will take its normal and nondeformed shape). To minimize any risk of stresses upon (and damage to) the polymer that will be affixed to the anchoring component, the polymer preferably should be affixed to an anchoring device while the anchoring device is in a nondeformed, non-stressed shape and size (there may be a few exceptions to that general rule, in highly specialized cases, such as if oscillating magnetic fields might be used to cause the device to undergo some type of shape-altering behavior after implantation, such as for sustained releasing of drugs). In addition, the complete implant device normally will be handled, stored, and shipped in its unstressed and undeformed shape and size, as established by the manufacturing process.

During surgical insertion, the implant (including both the shape-memory anchoring component, and the polymer component that is affixed to the anchoring component) will be squeezed into a second shape and size, which will have dimensions that will enable insertion of the implantable device, via a minimally-invasive incision, into a joint that is being repaired. In most circumstances, squeezing this type of flexible and shapeable device will cause it to become elongated in one direction, while temporarily assuming a narrower “width”. This will allow the device to be inserted into a joint, via a minimally-invasive incision, with assistance from an arthroscopic insertion tube if desired, thereby minimizing the amount of stresses and damage that will be inflicted on the tissues (including tiny blood vessels which are very important to proper recovery) that surround the insertion pathway.

After the insertion step has been completed, the “shape-memory” behavior of the anchoring component will cause it to return, as closely as possible (in view of any physical barriers or constraints it encounters and presses against), to its nondeformed shape and size. Even if the implant cannot return to exactly the same shape and size as the completely relaxed and nondeformed shape and size established by the manufacturing operation, the implant will nevertheless be allowed to return to a shape and size that will emulate the nondeformed manufactured shape and size.

Accordingly, to ensure that the implant will perform properly for years or decades, one of the goals of the design and manufacturing process for any implant will be to manufacture the implant in a controlled shape and size that are as close as possible to the final shape and size that the implant will be required to take, when it is being anchored to a hard bone surface or other tissue, during surgical implantation.

Accordingly, the anchoring rim for a femoral runner will be manufactured in a size and shape that will approximate (in a fully three-dimensional manner) the curved peripheral shape of a natural femoral runner. This can be aided by manufacturing such implants in a range of sizes, and allowing a surgeon to select a particular sized implant that most closely approximates the actual size of the femoral runner that must be replaced, in a particular patient. The anchoring rims for implants designed to replace tibial plateaus will be manufactured with entirely different shapes than for femoral runners, etc.

Returning to FIG. 1, callout number 120 indicates a layer of porous fabric, which will press directly against a bone surface after implantation. This fabric layer 120 will become an anchoring layer, which will promote the ingrowth of bony tissue, scar tissue, or other tissue into the implant, thereby creating (over a span of weeks, during recovery) a stronger and more secure anchoring of the implant to the bone or other tissue. In some implants, this goal might be accomplished by the same mesh component 180 that reinforces the hydrogel or other polymer (which may be, for example, a dual- or multi-layer mesh, or a mesh having a density, porosity, or other gradient). In other types of implants, a separate flexible anchoring layer that promotes tissue ingrowth may be preferred.

FIG. 1 also depicts an anchoring peg 150 (shown in more detail in FIGS. 5 and 7), a reinforcing mesh 180 (made of strong fibers embedded within polymeric material 185), and polymeric material 185, which has a smooth articulating surface 190.

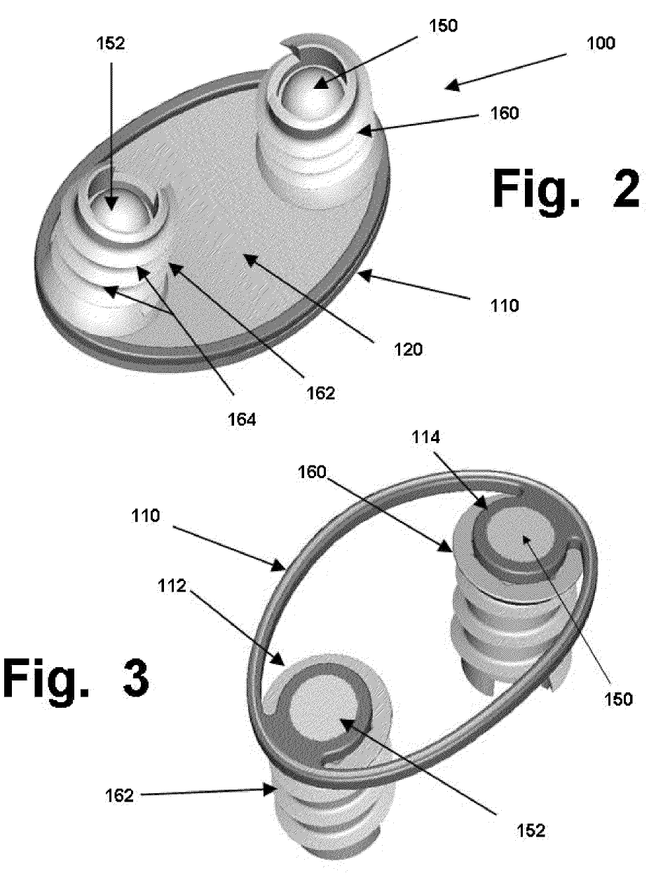

FIG. 2 depicts the same implant 100, showing the anchoring layer 120 and two anchoring pegs 150 and 152, surrounded by anchoring sleeves or receptacles 160 and 162, both of which have external threads 164. The anchoring receptacles 160 and 162 (which will be separate from the pegs and the implant, at the start of a surgical procedure) will be screwed into holes that have been drilled into a supporting bone, during the surgery, with the help of templates, bridges, guide-wires, and similar devices. After the receptacles 160 and 162 have been properly affixed in the holes drilled in the bone, the implant 100 will be inserted into the joint, such as through an insertion tube. It will be unrolled, expanded, and positioned properly, then the anchoring pegs 150 and 152 will be inserted into the anchoring receptacles 160 and 162.

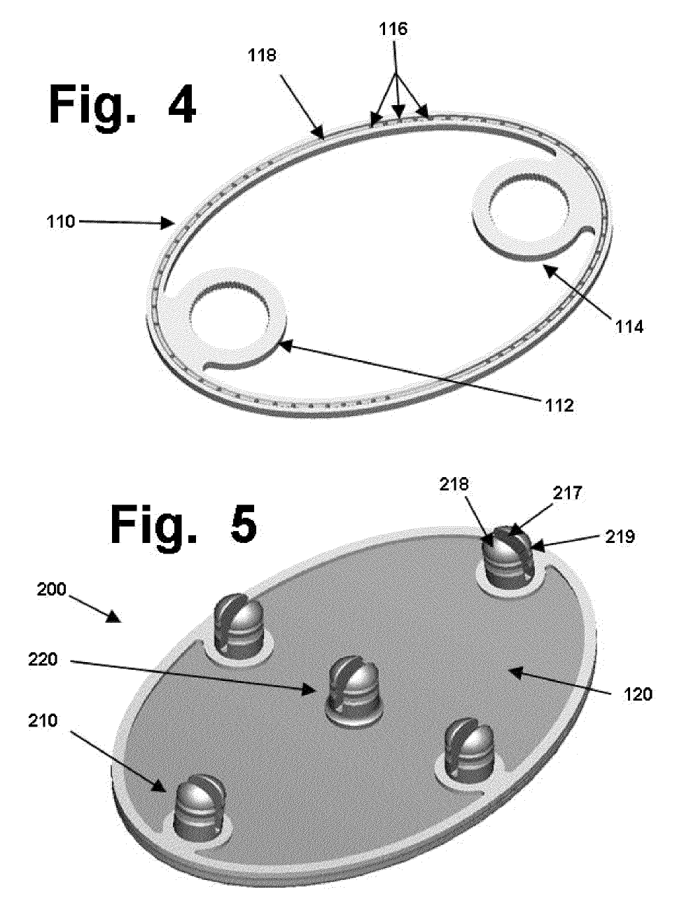

As illustrated more closely in FIG. 4, which shows an anchoring peg 220 in a larger view, the anchoring pegs have external surface ridges 212, which will engage accommodating ridges inside the “barrels” (i.e., the cylindrical inner surfaces, analogous to the barrel of a gun) of the anchoring receptacles. These interacting ridges on the pegs and receptacles will cause the pegs to become “locked” in the receptacles, once the pegs on an implant are pressed into the receptacles. The peg surface ridges 212 preferably should have generally “sawtooth” shapes, with small spacings (such as about ¼ to about 1/10 millimeters) between adjacent ridges. This will allow the pegs and receptacles to create a “ratcheting” engaging and locking mechanism.

FIG. 3 is a perspective view of implant 100 from an upper angle, showing the rim 110 in more detail, with peg attachment means 112 and 114 (shown as rings that will hold and secure the cylindrical upper ends of the pegs 150 and 152; any suitable coupling means can be used). The porous anchoring fabric 120 has been omitted from FIG. 3, since it sits on top of (and would hide) the peg attachment means 112 and 114. A layer of synthetic hydrogel polymer 185 (shown in FIG. 1) will sit on top of the porous anchoring fabric 120.

One embodiment of an anchoring rim 110 is illustrated in more detail in FIG. 4. Since it presumably will contain (and may even be made entirely of) a metallic alloy, such as nitinol, it can be provided with a plurality of stitching holes 116, to enable fabric layer 120 to be secured to rim 110. It can also be provided with a groove or depression 118 in the upper surface of rim 110, to hold strands of stitching fibers and/or any surplus fabric from fabric layer 120, thereby minimizing any risk of abrasion to a cartilage or hydrogel surface.

Alternately or additionally, two rings can be used that will fit together in a manner that will grab and secure a piece of fabric or other material, comparable to the types of hoops used in needlepoint.

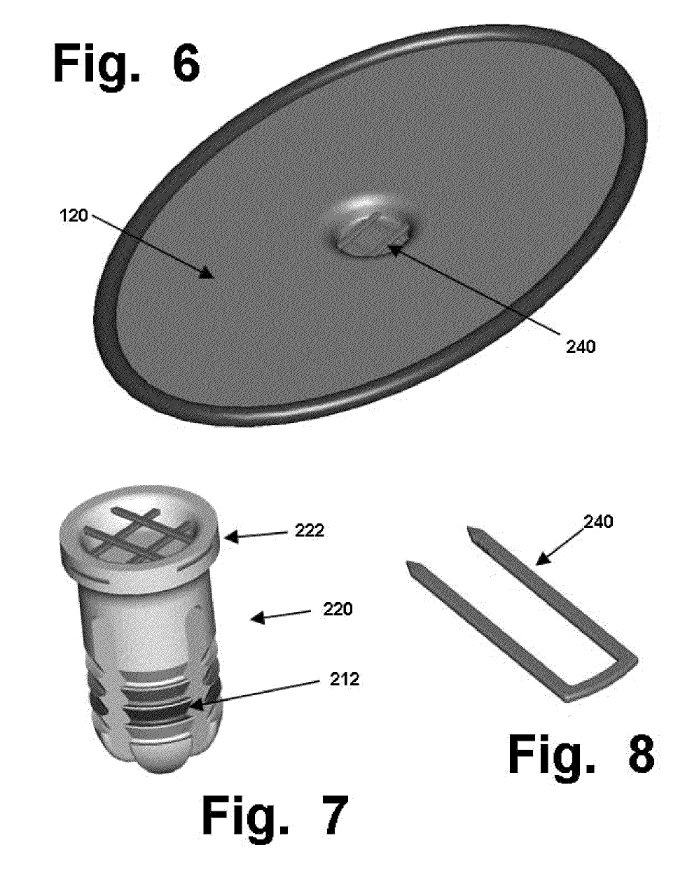

FIGS. 5-7 illustrate a design that will allow an internal anchoring peg 220 to be affixed to the porous fabric 120, in a manner that will not jeopardize a hydrogel layer that sits on top of the fabric 120. In the fully assembled implant, interior peg 220 has two relatively flat staples 240 passing through upper rim 222 of peg 220, as shown more clearly in FIG. 7. The two staples 240 pass through the upper rim 222 of internal peg 220 at offset heights, so that the two staples will not interfere with each other. As suggested by FIG. 6, staples 140 will be inserted into peg rim 222 after a small circular segment of the flexible porous fabric 120 has been pressed down into a depression (or dimple) in the uppermost surface of interior peg 220. The staples will penetrate and thereby engage and hold the fabric at that location, as indicated in FIG. 6.

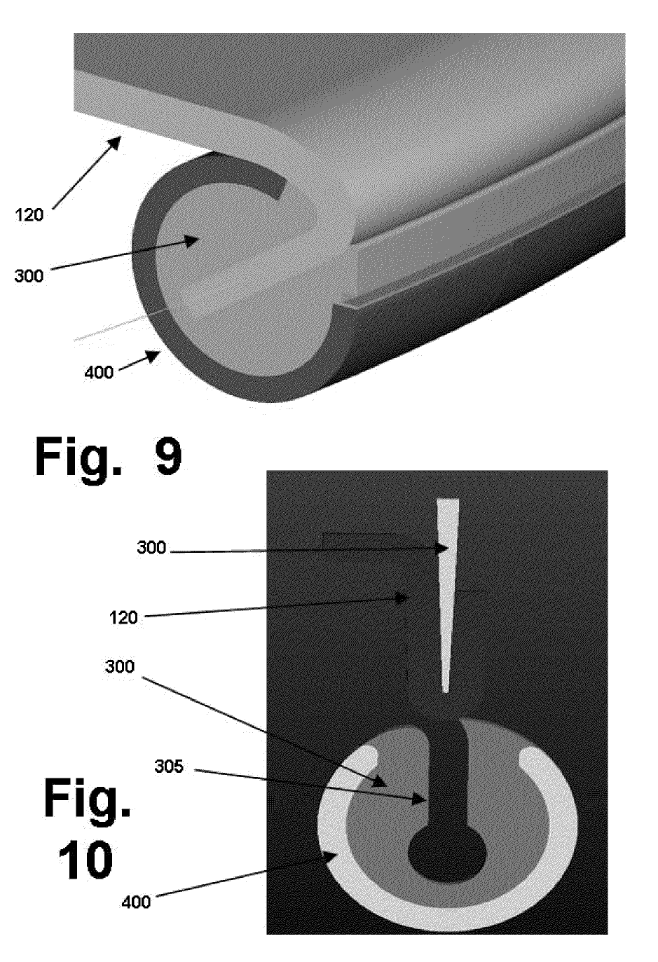

FIGS. 9 and 10 illustrate methods and designs for securing a porous anchoring fabric layer to a rim made of a metal alloy or hard plastic shape-memory material. In FIGS. 9 and 10, fabric layer 120 is gripped by a flexible and rubbery polymeric insert 300, which is squeezed and gripped by a generally circular rim component 400. FIG. 10 depicts the insertion of fabric 120 into a slot 305 in the polymeric insert 300, using an insertion tool 310. If desired, the polymeric insert 300 can be provided with a rounded or otherwise enlarged vacancy (or tunnel, etc.) at the bottom of slot 305, to hold a metallic “lock ring” that the cloth or other material 120 will be wrapped around.

As another alternative, the fabric or other material 120 can be positioned inside a metallic rim 400, and the polymeric insert can be injection-molded “in situ”, in a way that will cause the polymeric material to permeate through the fabric or other material 120 before the polymer sets and hardens. If desired, this approach can be supplemented and enhanced by machining or laser-cutting holes, slots, or other openings in one or more portions of a rim or hoop structure, thereby helping the polymeric material permeate more thoroughly throughout the interior volume inside the rim or hoop component.

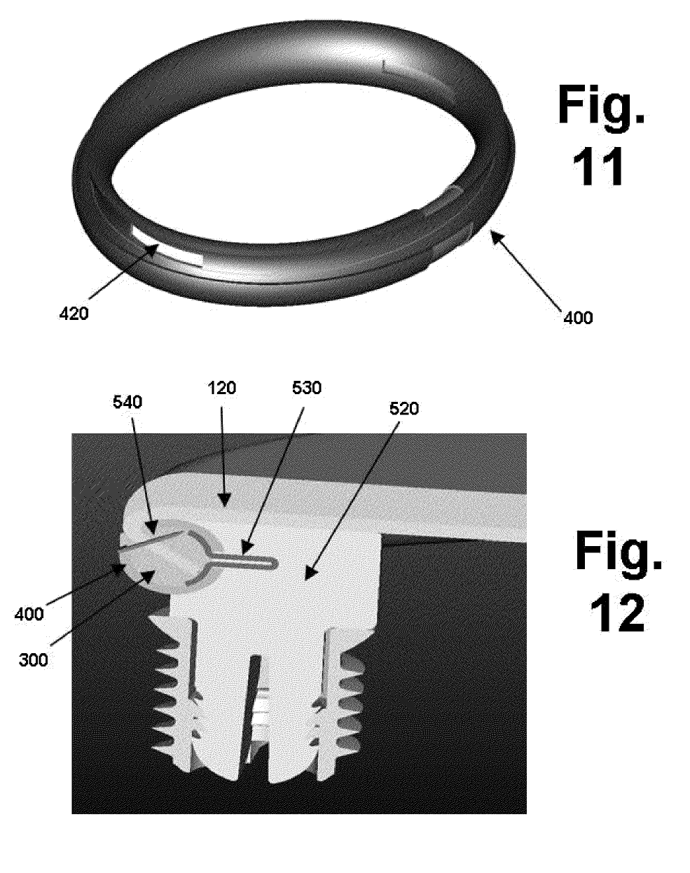

FIGS. 11 and 12 illustrate methods and components that can be used to affix a plurality of anchoring pegs or studs 520 to the rim component 400. In particular, FIG. 11 illustrates slots 420 in rim 400, which will engage ridges on anchoring pegs 520. FIG. 12 illustrates and describes a Y-shaped retaining clip 530 that can be passed through a rim slot 420, and positioned before the polymeric insert 300 is emplaced in the rim 400. Staple 540 can be passed through the fabric 120 and the polymer insert 300, to secure them in position.

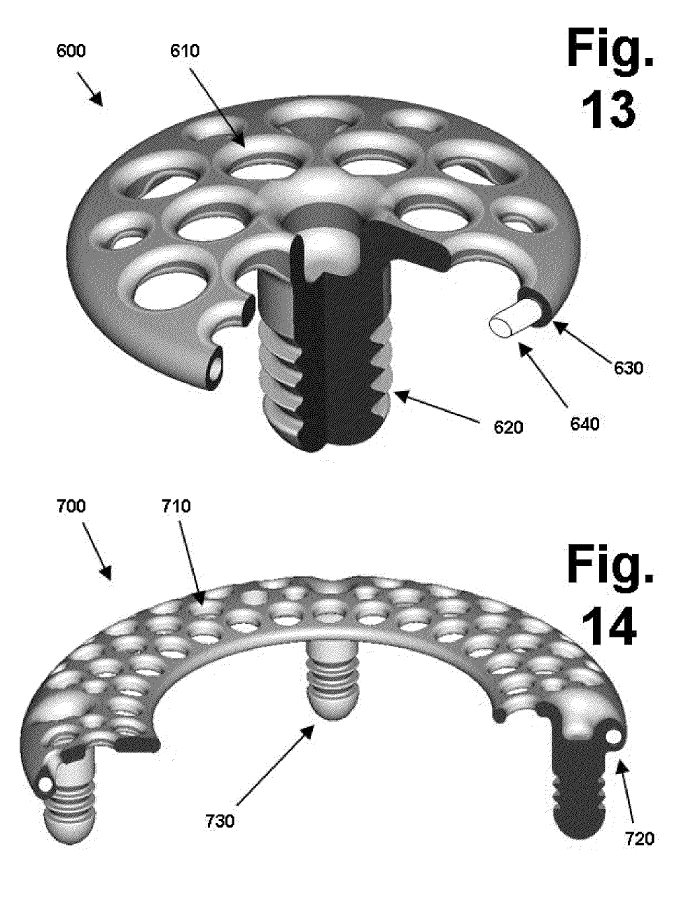

FIG. 13 illustrates a “button” implant 600, having a perforated “apron” 610 to which a reinforcing mesh and a hydrogel layer can be attached, and having a single anchoring peg 620. The rim 630 of implant 600 contains a shape-memory alloy wire 640.

A ring is not installed in the rim of an implant by pushing it into a tube or tunnel, after the implant has been molded. Instead, a preferred method of installation uses several polymeric spacers to hold the ring in position in a mold, spaced away from the walls of the mold. This allows the ring to be surrounded by the pre-polymer liquid that is poured into the mold, and when the pre-polymer liquid is cured into a solidified polymer, it surrounds and encloses the ring. The spacers used to support the ring in the mold should be made of a polymer that will bond properly with the polymer being used to form the support device.

Small “button” implants generally are designed for replacing either: (i) a small cartilage segment, such as in a finger or toe joint; or, (ii) only part of a larger cartilage segment such as a femoral runner or tibial plateau.

If used in a large joint, such as a knee, a button implant would need to be affixed, on a bone condyle, immediately adjacent to a segment of native cartilage on the same condylar surface. Unavoidably, some type of seam, juncture, or other interface must be created between the hydrogel surface of the implant, and the adjacent natural cartilage surface. No matter how carefully the seam is created, the resilient and slightly flexible nature of natural cartilage and synthetic hydrogels is likely to lead to some degree of intermittent flexing and separation between those two adjacent surfaces, as varying loads are imposed on them. When a joint such as a knee is subjected to various types of sliding and rotating pressures, loadings, and stresses, it is inevitable that the seam or other juncture between the implant surface and the adjacent native cartilage surface will undergo various moments when the two surfaces will be pushed, pulled, or otherwise flexed in slightly different directions, thereby creating momentary “gaps” between the two adjacent surfaces.

In most patients who need cartilage repair, the risk is high that over a span of years or decades, such gaps, even if they occur only sporadically and momentarily, will eventually cause some level of abrasion of (and gradual damage to) an opposing and articulating cartilage surface that an implant rubs and slides against.

When that factor is taken into account, the likelihood is high that in most people who are not extremely aged and who hope to walk again without a cane or other support, the preferred form of implant will be a “complete segment” implant, which will replace a complete femoral runner, tibial plateau, patellar segment, or other cartilage segment. Unlike “button” implants that unavoidably create a seam with a tiny but potentially abrasive gap between the implant and the native cartilage, “complete segment” implants can provide a consistent and smooth surface across the entire surface of the implant, without any seams, junctures, or gaps. Accordingly, such implants generally should be regarded as preferable for most patients, and one of the features of this invention is that it can be used to manufacture “complete segment” implants that can replace: (1) complete femoral runners, tibial plateaus, or patellar surfaces, in knee joints; (2) complete ball-head and socket-surface segments, in hip or shoulder joints; and, (3) entire knuckle or similar surfaces, in finger, hand, toe, foot, or ankle joints.

That statement needs clarification, with regard to femoral runners and tibial plateaus. In a mammalian knee joint, two parallel femoral runners are present, side-by-side, on the medial (interior) and lateral (exterior) sides of the knee. The cartilage segment at the bottom of a femur includes both femoral runners as well as a pateller (knee cap) portion on the front surface of the bone. However, because of how knee cartilage is shaped in humans, orthopedic surgeons often perform a “unicompartmental” repair of just one runner, without having to also replace the other runner in a “bicompartmental” operation (or the patellar segment as well, in a “tricompartmental” operation).

Regardless of whether a knee repair will be unicompartmental or bicompartmental, implants as disclosed herein can be manufactured with essentially any desired size and shape (including curved shapes, such as to replace a curved femoral runner), without having any seams or gaps on a surface that will be subjected to loading, wear, and potential abrasion during the years following the operation.

In addition, hydrogel implants with supporting components as disclosed herein can be adapted to replace meniscal or labral wedges in knee, hip, and shoulder joints, using designs that will become apparent to orthopedic surgeons and others who design and manufacture such implants.

Accordingly, support device 700, shown in FIG. 14, can be referred to as large support, a hoop support, a “complete segment” support, or any other suitable term. It is illustrated as a circular open hoop to make the drawings easier and faster to create, handle, and transmit, using computers. In actual use, most such implants will emulate the size and shape of a cartilage segment being replaced, such as a femoral runner or tibial plateau, some implants will have non-planar shapes (such as curved implants that will conform to the rounded surfaces of femoral runners), and there usually will not be an open or vacant area in the middle of the implant.

Support device 700 comprises an apron component 710 with numerous openings and an outer rim 720, and several spaced anchoring pegs 730 located near the outer rim 720. All of these components perform functions similar to those described above, for button implants.

Thus, there has been shown and described a new and useful means for creating supporting and anchoring systems that can enhance and strengthen hydrogel implants, for replacing damaged cartilage and for other surgical and medical uses. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

1. A surgically implantable device, comprising:
a. at least one first part made from a shape-memory material; and,
b. at least one second part made from a polymer material,
wherein said device is designed for surgical replacement of cartilage in a mammalian joint.

2. The implantable device of claim 1 wherein said first part is formed as a rim that has been secured to at least one peripheral edge of said second part and that is provided with means for anchoring said rim to a bone.

3. The implantable device of claim 2 wherein said means for anchoring said rim to a bone are selected from the group consisting of: (i) a plurality of pegs, affixed to said rim and designed for insertion into accommodating anchoring devices that can be emplaced in a bone surface prior to arthroscopic insertion of the implantable device; and, (ii) means for affixing to said rim a plurality of pegs designed for insertion into accommodating anchoring devices that can be emplaced in a bone surface.

4. The implantable device of claim 1 wherein said first part is formed as a polymer-supporting component affixed to a single peg that is designed for insertion into an accommodating anchoring device that can be emplaced in a bone surface prior to arthroscopic insertion of the surgically implantable device.

5. The implantable device of claim 1 wherein said first part made from a shape-memory material has dimensions that are designed to provide:
a. a first nondeformed shape and size that were established by a manufacturing process;
b. a second narrowed shape and size having dimensions that will enable insertion of the implantable device, via a minimally-invasive incision, into a joint that is being repaired; and,
c. a third shape and size that emulates the first nondeformed shape and size.

6. The implantable device of claim 1 wherein said polymer material is a flexible polymeric component affixed to said first part.

7. The implantable device of claim 1 wherein said flexible polymeric component is a synthetic hydrophilic polymer that becomes a flexible hydrogel when hydrated with saline solution.

8. The implantable device of claim 1 wherein a layer of porous material that promotes tissue ingrowth is affixed to said first part.

9. The implantable device of claim 2 wherein a layer of porous material that promotes tissue ingrowth is affixed to said rim and is completely surrounded by said rim.

10. The implantable device of claim 8 wherein said second part made from a polymer material is affixed to said layer of porous material that promotes tissue ingrowth.

11. The implantable device of claim 8 wherein said second part made from a polymer material is affixed to said layer of porous material by means of a fibrous mesh that is embedded within said second part and that is also affixed to said layer of porous material.

12. The implantable device of claim 8 wherein said layer of porous material that promotes tissue ingrowth is affixed to said first part by means comprising mechanical gripping of an elastomeric material that surrounds a portion of said porous material, by said first part.

13. The implantable device of claim 1 wherein said polymer material has been given a negative surface charge and is suited for replacing hyaline cartilage in at least one type of mammalian joint.

14. A surgically implantable device, comprising:
a. at least one first part made from an elastic material that is designed to allow insertion of the implantable device into a mammalian joint in a deformed shape via a minimally-invasive incision, and to then return to a nondeformed shape within the joint; and,
b. at least one second part made from a synthetic polymer,
wherein said device is designed for surgical replacement of cartilage in a mammalian joint.

15. The implantable device of claim 14 wherein said first part is formed as a rim that has been secured to at least one peripheral edge of said second part and that is provided with means for anchoring said rim to a bone.

16. The implantable device of claim 14 wherein said first part is formed as a polymer-supporting component affixed to a single peg that is designed for insertion into an accommodating anchoring device that can be emplaced in a bone surface prior to insertion of the surgically implantable device.

17. The implantable device of claim 14 wherein said first part made from an elastic material has dimensions that are designed to provide:
a. a first nonstressed shape and size that were established by a manufacturing process;
b. a second narrowed shape and size having dimensions that will enable insertion of the implantable device, via a minimally invasive incision, into a joint that is being repaired; and,
c. a third shape and size that emulates the first nonstressed shape and size.

18. The implantable device of claim 14 wherein said polymer material is a flexible polymeric component affixed to said first part.

19. The implantable device of claim 14 wherein said flexible polymeric component is a synthetic hydrophilic polymer that becomes a flexible hydrogel when hydrated with saline solution.

20. The implantable device of claim 14 wherein a layer of porous material that promotes tissue ingrowth is affixed to said first part.

21. The implantable device of claim 14 wherein said second part made from a polymer material is affixed to said layer of porous material that promotes tissue ingrowth.

22. The implantable device of claim 14 wherein said second part made from a polymer material is affixed to said layer of porous material by means of a fibrous mesh that is embedded within said second part and that is also affixed to said layer of porous material.

23. The implantable device of claim 14 wherein said synthetic polymer has been given a negative surface charge and is suited for replacing hyaline cartilage in at least one type of mammalian joint.

24. A surgically implantable device, comprising:
a. at least one first part made from an elastic material that is designed to allow insertion of the implantable device into a mammalian recipient in a deformed shape, via an arthroscopic insertion tube, and to then return to a nonstressed shape within the joint, in a manner that will enable permanent anchoring of the implantable device to bone or tissue in the mammalian joint;
b. at least one second part made from a flexible polymer;
c. at least one tissue anchoring layer comprising a porous layer of material that promotes tissue ingrowth into said layer of material, after surgical implantation;
d. means for securing said tissue anchoring layer to said first part; and,
e. means for securing said tissue anchoring layer to said second part.

25. The implantable device of claim 24 wherein a fibrous reinforcing mesh is embedded within at least a portion of the flexible polymer of said second part, and wherein said fibrous reinforcing mesh is secured to said tissue anchoring layer.

26. The implantable device of claim 24 wherein said flexible polymer comprises a synthetic hydrogel polymer.

27. The implantable device of claim 24 wherein said flexible polymer has been given a negative surface charge and is suited for replacing hyaline cartilage in at least one type of mammalian joint.

28. A surgically implantable device, comprising:
a. at least one first part made from a shape-memory material; and,
b. at least one second part made from a soft polymer that is suited for protecting and nurturing transplanted cells.

29. The surgically implantable device of claim 28, wherein the soft polymer is made from a synthetic hydrophilic polymer that becomes a flexible hydrogel when hydrated with saline solution.

30. The surgically implantable device of claim 28, wherein the second part made from a soft polymer is enclosed within two or more porous layers affixed to a shape-memory anchoring device.
